(19) United States
(12) Patent Application Publication
(10) Pub. No.: US 2005/0137609 A1
(43) Pub. Date: Jun. 23, 2005

(54) Universal cardiac introducer

(76) Inventor: Gerald Guiraudon, London (CA)

Correspondence Address: RICHES, MCKENZIE & HERBERT, LLP, SUITE 1800, 2 BLOOR STREET EAST, TORONTO, ON, M4W 3J5, CA

(21) Appl. No.: 10/736,786

(22) Filed: Dec. 17, 2003

(51) Int. Cl. A61F 11/00

(52) U.S. Cl. 606/108

(57) ABSTRACT

The present invention may be characterized as a heart access manifold having a manifold wall and at least one exit port and one entry port. The exit port through the manifold wall is adapted to be sealably engaged about an opening in the wall of a heart for communication with the interior of the heart and with the interior of the manifold to be under the same pressure as the interior of the heart. At least one entry port is provided through the manifold wall to provide access through the manifold wall into the interior heart via the exit port. Each entry port is sealable to maintain pressure on an interior side of the manifold wall. The entry port is adapted for passage therethrough to the interior side of the manifold wall of at least one implement in sealed relation so as to maintain pressure on the interior side of the manifold. The implement may be one which is selected to perform any operation that is desired or necessary within the interiors of the manifold or the heart. Suitable implements include microwave or ultrasonic probes, knives, cutters, staplers, holders, clamps, suturing devices, lasers and the like which are useful for carrying out procedures within the interior of the heart.

### SCOPE OF THE INVENTION

This invention relates to a device for accessing the interior of the heart, to the use of the same and to methods for heart surgery.

### BACKGROUND OF THE INVENTION

The majority of cardiac surgery is performed with a stopped (non-beating) heart. This approach is very successful, but is very expensive and has a high complication rate. Recently, beating-heart surgery has been developed to treat coronary stenosis. Bypass of coronary arteries is amenable to this technique since the coronary arteries are located on the outside (epicardial) surface of the heart. However, there are currently no techniques that permit beating heart surgery to be performed on structures inside the heart, or on the inside (endocardial) surface of the heart. Such techniques would allow faster, less expensive, and safer cardiac surgery for these conditions.

### SUMMARY OF THE INVENTION

The present invention provides a Universal Cardiac Introducer (UCI) that facilitates off-pump, beating-heart surgery for conditions that require repair or modification to the interior of the heart. The UCI permits access to any chamber of the beating heart. It allows for the insertion and manipulation of common, as well as specially designed Surgical Instruments and Tools. These instruments can be visualized and manipulated under ultrasound or other imaging techniques, and with the assistance of robotic techniques, to perform valve replacement or repair, atrial fibrillation ablation, congenital repairs and the like.

The UCI or cardiotomy access adapter in one preferred embodiment consists of a flexible sleeve that attaches to the beating heart, preferably, the epicardial surface of the heart. Various adaptors can be used to accommodate standard surgical instruments, such as forceps, scissors, etc. Other adaptors may accommodate specially designed surgical instruments or tools. Specially designed surgical instruments in accordance with this invention include a mitral valve repair tool device and an atrial fibrillation ablation adapter.

The present invention provides the UCI as a manifold to access the heart and methods of use which overcome difficulties arising in beating heart surgery in the interior of the heart including the following difficulties:

Control of bleeding. Because an access opening into the heart exposes the full cardiac blood pressure, and patients will typically be pre-treated with anticoagulants, the preferred UCI permits bleeding to be controlled during application of the UCI to the heart and during the introduction, manipulation and removal of instruments.

Flexibility. Since a beating heart will be moving, the preferred UCI is adapted to be flexible enough to prevent excessive mechanical stress or on the heart tissues or the instruments.

Versatility. The UCI accommodates many types of surgical instruments, and allows for the easy manipulation of these instruments. The UCI permits several instruments to be introduced simultaneously. The UCI accommodates robotic instruments and tools.

Visualization. The UCI permits adequate visualization using ultrasound, MRI, or other imaging techniques.

Clotting. The UCI is adapted to minimize the potential for blood clots and other emboli.

Safety. The preferred UCI is designed to ensure that the opening and closure/repair of the cardiac port be simple, fast and effective. The UCI preferably provides a secondary method of occluding the system to prevent blood loss in case the primary system fails.

The present invention may, in one aspect, be characterized as a sealed chamber is provided external to the heart and in communication with the interior of the heart. If the heart is a beating heart, the interior of the cavity is under the same pressures as the interior of the heart. The chamber is in communication with the heart via an entry port from the chamber which entry port is sealed to the heart and secured to the structure of the heart as to the wall of the heart about an opening through the heart wall. Usual entry ports are in the left and right atrial appendages and the left and right ventricles. Securing may be accomplished either to the outside surface of the heart wall or to the inside surface of the heart wall or both by any suitable arrangement.

Access is provided into the sealed chamber where the sealed chamber is exterior of the heart with access being provided by entry ports which are sealable to maintain the pressure within the sealed chamber. Arrangements are made for implements to be inserted into the sealed chamber and via the sealed chamber into the interior of the heart for manipulation of the instruments to perform operations on the heart within the sealed chamber and within the interior of the heart while maintaining the sealed chamber enclosed to maintain blood pressure of the heart within the chamber. Various mechanisms can be provided for sealing different portions of the enclosed chamber such that other portions of the chamber which become isolated from the heart may be opened as to the atmosphere to permit initial insertion and removal of instruments. Instruments which are to pass through the enclosed chamber are preferably adapted for manipulation to various positions within the chamber and within the interior of the heart to carry out desired procedures.

The particular volume of the sealed chamber is not limited when, on one hand, it may have minimal volume and, on the other hand, the enclosed chamber could have substantial volume and it could, for example, extend from the heart as a relatively elongate tube.

The nature of the enclosure wall which encloses the sealed chamber is not limited, it may be flexible or rigid. The wall may comprise a number of removable and separable components. The wall may provide a number of branches which may individually be sealed and opened.

The present invention, in another aspect, may be characterized as a heart access manifold having a manifold wall and at least one exit port and one entry port. The exit port through the manifold wall is adapted to be sealably engaged about an opening in the wall of a heart for communication with the interior of the heart and with the interior of the manifold to be under the same pressure as the interior of the heart. At least one entry port is provided through the manifold wall to provide access through the manifold wall into the interior heart via the exit port. Each entry port is sealable to maintain pressure on an interior side of the manifold wall. The entry port is adapted for passage therethrough to the interior side of the manifold wall of at least one implement in sealed relation so as to maintain pressure on the interior side of the manifold. The implement may be one which is selected to perform any operation that is desired or necessary within the interiors of the manifold or the heart. Suitable implements include microwave or ultrasonic probes, knives, cutters, staplers, holders, clamps, suturing devices, lasers and the like which are useful for carrying out procedures within the interior of the heart.

The manifold wall preferably has a main sleeve portion to define a main conduit providing communication from the entry port to the exit port. Preferably, a closure mechanism is provided to sealably close the main conduit against communication therethrough. A closure mechanism may comprise merely the manifold with the main sleeve being flexible and adapted to be closed as by suture or clamp which may be separate or integrally incorporated into the manifold wall. The manifold wall may also be adapted to assume either a biased open position or a biased closed position.

For each entry port, preferably, a branch sleeve portion is provided which defines a branch conduit for communication from the entry port into the interior of the heart, preferably, through the main conduit defined in the main sleeve portion. Preferably, a closure mechanism may be provided to sealably close each branch sleeve portion against communication therethrough. The closure mechanism may constitute automatically reclosable valves such as a bi-cuspid valve or a simple flap valve. Many different devices may be used as a closure mechanism for the branch conduits. The closure mechanism may also comprise merely the provision of the branch sleeve portion as a flexible sleeve which can be closed by a simple clamp or string suture.

The main sleeve portion may have an exit end about the exit port and entry end opening to the branch sleeve portions. The entry end may be closed by a distribution wall or distribution cap carrying a branch port therethrough for each branch sleeve portion with each branch port opening into an exit end of its respective branch sleeve portion.

Preferably, a cuff is provided at the exit end of the main sleeve portion for sealable engagement to the wall of a heart about the opening through the wall of the heart. Preferred means for sealing the cuff to the heart include suturing although various other arrangements could be provided as, for example, by providing resealable clamping to the wall of the heart about the opening and receiving the wall of a heart inside an expandable annular cup.

Instruments are provided to extend through the manifold, through an opening in the wall of the heart and into the interior of the heart. The instruments are to be slidably movable into and out of the opening to the heart and may preferably be slidable relative to the entry port in the branch sleeve while maintaining a sealed relation thereto. Insofar as the main sleeve portion and the branch sleeve portions may be flexible and, for example, extendable and collapsible or otherwise resilient, then the instruments may be movable relative to the heart by flexure of the main sleeve portion and the branch sleeve portions.

The heart access manifold may comprise a number of different separate elements which can be removably coupled together. For example, the main sleeve portion may be a separate element removable from a distribution cap carrying the branch sleeves such that different distribution caps can be applied and/or replaced carrying different instruments. As well, each end of each branch sleeve may be replaceable as, for example, to provide a replaceable entry port adapted for engaging about different sized instruments. Each branch sleeve may also be coupled to sub-branch sleeves such that a plurality of sub-branch sleeves may extend into one branch sleeve and, hence, into the main sleeve. When a removable distribution cap is to be applied, preferably, the main sleeve may be closed.

Preferably, the main sleeve portion may be coupled to the heart with an annular ring portions disposed between the main sleeve portion and the heart which is elastic and permits movement of the heart without the main sleeve portion moving to the same extent.

An entry port may be provided to vent air, as after filling the manifold with blood. Alternately, air may be vented from the interior of the manifold as by using a needle.

In one aspect, the present invention provides a heart access manifold having an interior within a manifold wall, an exit port through the manifold wall and at least one entry port through the manifold wall,
the exit port being adapted to sealably engage about an opening in a wall of the heart for communication with the interior of the heart to place the interior of the manifold to be under the same pressure as the interior of the heart,
the at least one entry port providing access through the manifold wall into communication with the interior of the heart via the exit port, each entry port sealable to maintain pressure on the interior side of the manifold,
each entry port adapted for passage therethrough to the interior of the manifold of at least one implement while maintaining pressure in the interior of the manifold.

In another aspect, the present invention provides a method of heart surgery on a beating heart by access to the interior of the heart via an enclosed chamber in communication with the interior of the heart.

### BRIEF DESCRIPTION OF THE DRAWINGS

Further aspects and advantages of the present invention will become apparent from the following description taken together with the accompanying drawings in which:

FIG. 1 is a perspective view of a heart access manifold in accordance with a first embodiment of the present invention;

FIG. 2 is a cross-sectional side view of the heart access manifold of FIG. 1 along section line 2-2′;

FIG. 3 is a bottom view of the heart access manifold of FIG. 1;

FIG. 4 is a schematic partially cross-section view of a heart with a portion of a wall of the heart clamped;

FIG. 5 is a schematic side view as seen in FIG. 4, however, with the heart access manifold in accordance with FIG. 1 secured thereto;

FIG. 6 is a schematic view similar to that in FIG. 5 but with instruments secured in each of the branch sleeve portions of the heart access manifold and with the heart opening closed;

FIG. 7 is a schematic side view of the heart as seen in FIG. 6 but with the heart opening closed and instruments within each of the branch sleeve portions;

FIG. 8 is a schematic side view similar to that in FIG. 7, however, with instruments extended through the wall of the heart into the interior of the heart;

FIG. 9 is a pictorial view showing the heart and the heart access manifold with instruments extending thereinto as would be seen externally in the conditions shown in both FIG. 7 and FIG. 8;

FIGS. 10 and 11 are schematic cross-sections similar to that in FIG. 8, however, with the instruments withdrawn into the branch sleeve portions and with the main sleeve portion closed;

FIG. 12 is a schematic side view similar to that in FIG. 5 but without the wall of the heart clamped;

FIG. 13 is a schematic side view similar to FIG. 12 with instruments in each branch sleeve;

FIGS. 14 and 15 illustrate one instrument received within an extendable branch sleeve which is extended in FIG. 15;

FIG. 16 illustrates an instrument received in an inverted branch sleeve;

FIG. 17 shows a schematic cross-section of an end of a branch sleeve with an end portion of an instrument received therein showing, firstly, a sealing arrangement between the branch sleeve and the instrument and, secondly, a valve to close the branch sleeve;

FIG. 18 shows a view similar to FIG. 17 of a different branch sleeve;

FIG. 19 illustrates a second embodiment of a heart access manifold in accordance with the present invention;

FIG. 20 illustrates a schematic partially sectioned side view of a heart with the heart access manifold in accordance with the second embodiment of FIG. 19 secured thereto;

FIG. 21 illustrates a third embodiment of a heart access manifold in accordance with the present invention similar to that shown in FIG. 19 and including a removable closure plug;

FIG. 22 illustrates an exploded view of a heart access manifold in accordance with a fourth embodiment of the present invention in an inflated condition;

FIG. 23 illustrates a main sleeve portion of the heart access manifold of FIG. 22 in a collapsed, closed condition;

FIG. 24 is a schematic, cross-sectional side view showing an arrangement for quick coupling of the upper end of the main sleeve portion of the heart access manifold of FIG. 22 with a distribution cap;

FIG. 25 schematically illustrates a cross-sectional side view showing a preferred configuration for a cuff of a heart access manifold in accordance with a fifth embodiment of the present invention;

FIG. 26 is a cross-sectional side view similar to that in FIG. 22 but of a sixth embodiment of a heart access manifold;

FIG. 27 illustrates in a schematic pictorial view a holding loop adapted to be secured to a cuff of a heart access manifold;

FIG. 28 is a schematic cross-sectional side view of a heart access manifold in accordance with the present invention as receiving an instrument comprising a cutting tool for cutting an opening in a wall of the heart;

FIG. 29 is a side view of the blade of FIG. 28;

FIGS. 30 to 34 are respective schematic side views of the cutting instrument as shown in FIG. 23 in successive positions in use for cutting an opening in a wall of the heart;

FIG. 35 is a pictorial view of an instrument for use with a heart access manifold in accordance with the present invention to repair Mitral valves using the Mitral valve repair device;

FIG. 36 is a pictorial view of the Mitral valve repair device shown in FIG. 35 in an expanded condition;

FIG. 37 is a pictorial view of the Mitral valve repair device of FIG. 36 in a collapsed condition;

FIG. 38 illustrates a schematic pictorial view of the Mitral valve repair device of FIGS. 36 and 37 in a collapsed condition with a plurality of flexible tubes secured thereto and extending axially within a tube of the instrument of FIG. 35;

FIG. 39 is a schematic cross-sectional side view of a heart to which a heart access manifold in accordance with the present invention has been coupled and showing instruments including a Mitral valve repair instrument for use in applying a Mitral valve repair device similar to that of the type illustrated in FIGS. 35 to 38;

FIG. 40 shows a view substantially the same as that shown in FIG. 39, however, illustrating the application of the Mitral valve repair device to a Mitral valve;

FIG. 41 illustrates a loop holding instrument for use with a heart access manifold in accordance with the present invention;

FIG. 42 is a schematic pictorial partially cross-sectioned view of a further embodiment of a heart access manifold in accordance with the present invention carrying a loop holder as shown in FIG. 41 with the loop disposed within the interior of the heart.

### DETAILED DESCRIPTION OF THE DRAWINGS

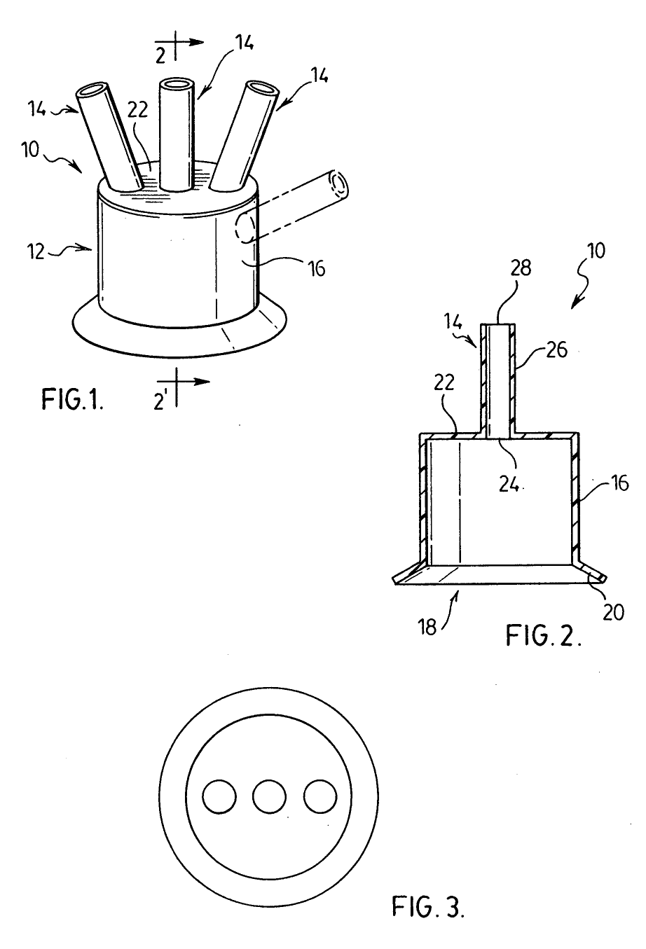

Reference is made first to FIGS. 1, 2 and 3 which illustrate a first embodiment of a heart access manifold 10 in accordance with the present invention. The manifold 10 has a main sleeve 12 from which three branch sleeves 14 extend. The main sleeve 12 is formed by a cylindrical side wall 16 open at one end as exit port 18. A cuff 20 is disposed as a radially outwardly extending annular flange about the exit port 18. The other end of the main sleeve 12 is closed by a distribution wall 22 having three branch ports 24. Each branch sleeve 14 has a cylindrical branch side wall 26 coupled about its respective branch port 24. Each branch sleeve 14 is open at a branch entry port 28.

In the first embodiment, the entirety of the manifold 10 is preferably formed from a flexible, fluid impermeable fabric-like material, preferably having Heparin (trade mark) binding or some other similar binding to at least assist in preventing clotting. The cuff 20 may preferably comprise Dacron (trade mark) material.

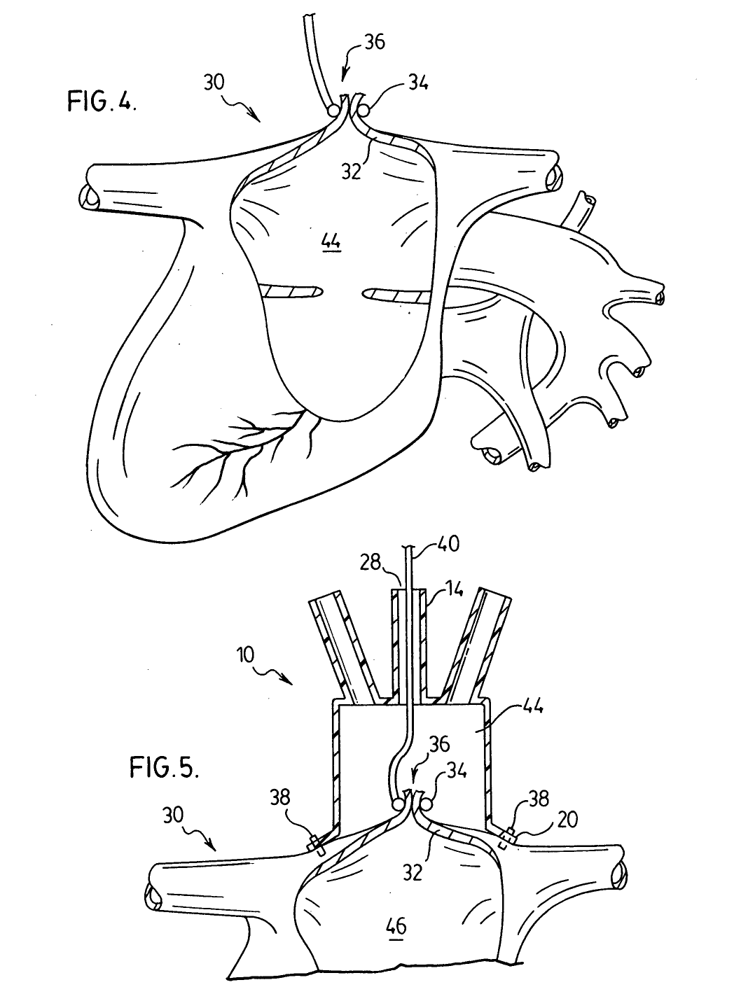

Use of the heart access manifold 10 in accordance with FIGS. 1 to 3 is now briefly described with reference to FIGS. 4 to 10. While not shown in the Figures, in a preferred procedure, the heart of a patient is exposed by a standard, minimally invasive direct coronary artery bypass procedure under general anaesthesia in which the patient's rib cage is divided to provide access to the heart.

FIG. 4 shows a partially sectioned view of a heart 30 showing that a portion of the wall 32 of the heart 30 has been clamped in a purse-string suture 34 and, after clamping, has been cut so as to provide a cardiac port 36 through the side wall of the heart.

As seen in FIG. 5, a heart access manifold 10, as shown in FIG. 1, is secured to the epicardial surface of the wall 32 of the heart by securing the cuff 20 to the wall 32 of the heart with schematically shown sutures 38. The heart access manifold 10 is secured so as to have its exit port 18 extend circumferentially about the cardiac port 34.

The end string 40 of the purse-string suture 34 is passed outwardly through the heart access manifold 10 as, for example, to extend out one of the branch sleeves 14 and to be accessible from the entry port 28 of that branch sleeve.

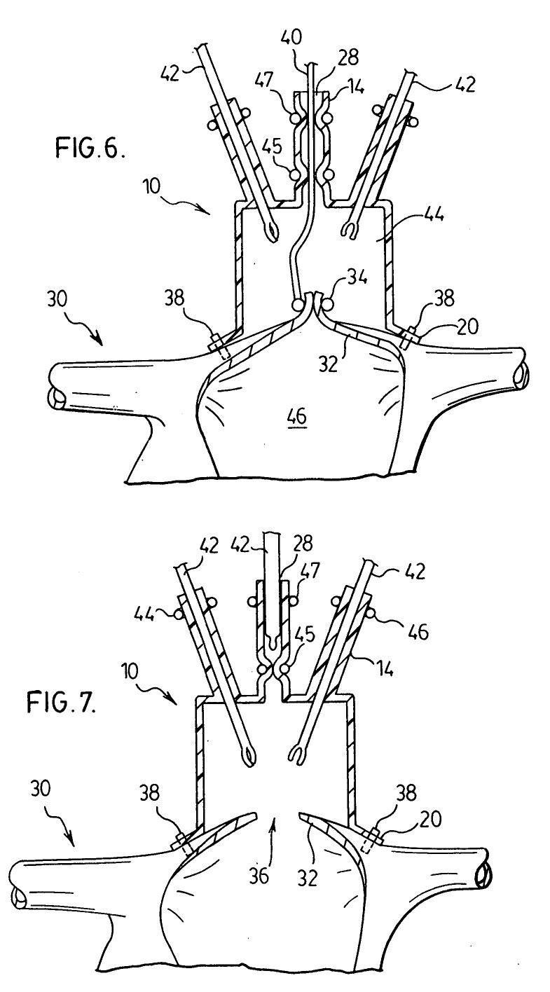

As seen in FIG. 6, two instruments 42 are introduced into two of the branch sleeves 14 and each of the branch sleeves 14 are sealed thereby by purse-string sutures or clamps 44, 45 and 46. In the condition of FIG. 6, the purse-string suture 34 holding the cardiac port 36 closed is pulled to remove suture 34 and to open the cardiac port 36 so as to permit the interior 44 of the manifold to be in communications with the interior 46 of the heart and at the same pressure thereof, as illustrated in FIG. 7. A fluid impermeable seal is formed between the heart access manifold 10 and the wall 33 of the heart by reason of the sutures 38 holding the cuff 20 to the wall of the heart. Each of the branch sleeves 14 are secured in a sealed manner, as by their respective clamps 44.

In FIGS. 6 and 7, two clamps 44 and 46 seal the branch sleeves 14 onto the extension of the instruments 42. Clamps 45 and 47 seal the middle branch sleeve 14 collapsed onto the string 40 in FIG. 6. After the string 40 is removed, the clamps 45 and 47 may be used to assist in controlled release of air from the interior 44 of the manifold as blood from the heart fills the manifold. FIG. 7 shows, after the string 40 has been removed, a third instrument 42 introduced into the middle branch sleeve 44 and sealed therein by clamp 47.

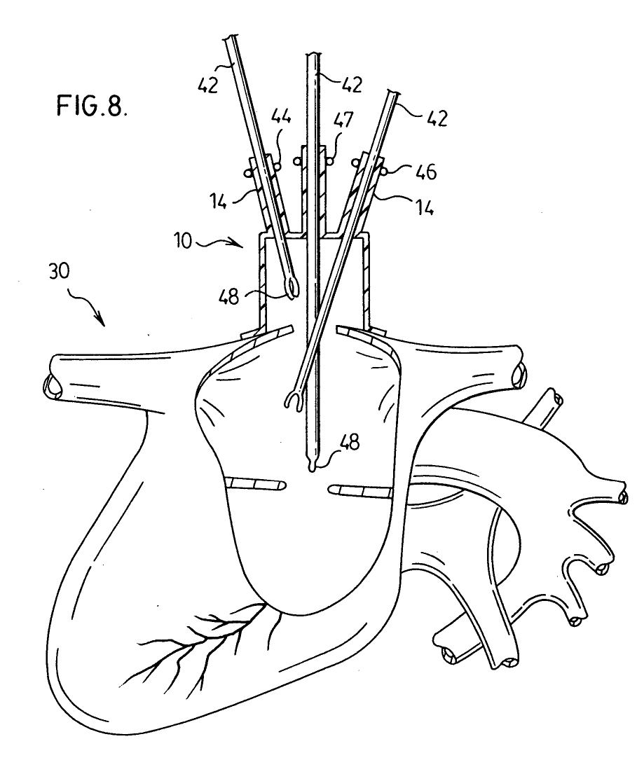

FIG. 8 illustrates the condition in FIG. 7 but with clamp 45 removed, the three instruments 42 be moved to positions in which their inner ends 48 are received within the interior of the manifold 10 and/or in the interior of the heart as shown in FIG. 8. In accordance with the present invention, the instruments 42 are preferably, slidably received in a sealed manner within each of the entry ports to the branch sleeves 14.

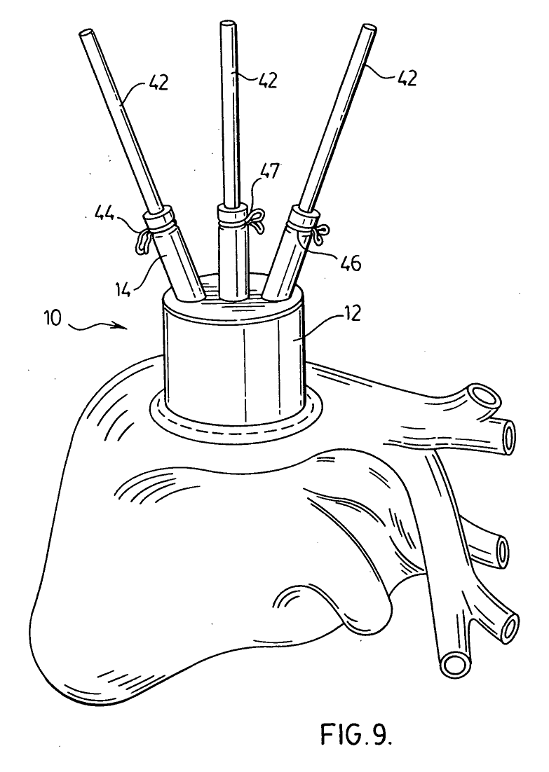

FIG. 9 illustrates a pictorial view of the instruments 42 as extending from the manifold 10 when inflated and applied to a heart as arises in the condition of FIGS. 7 and 8. In this condition, the various instruments 42 are adapted for manipulation and may be moved and manipulated within the interior of the manifold and within the interior of the heart.

One method of inserting or ending an instrument 42 has been illustrated with reference to FIGS. 6 and 7 by closing one of the branch sleeves. As another method, the main sleeve may be closed.

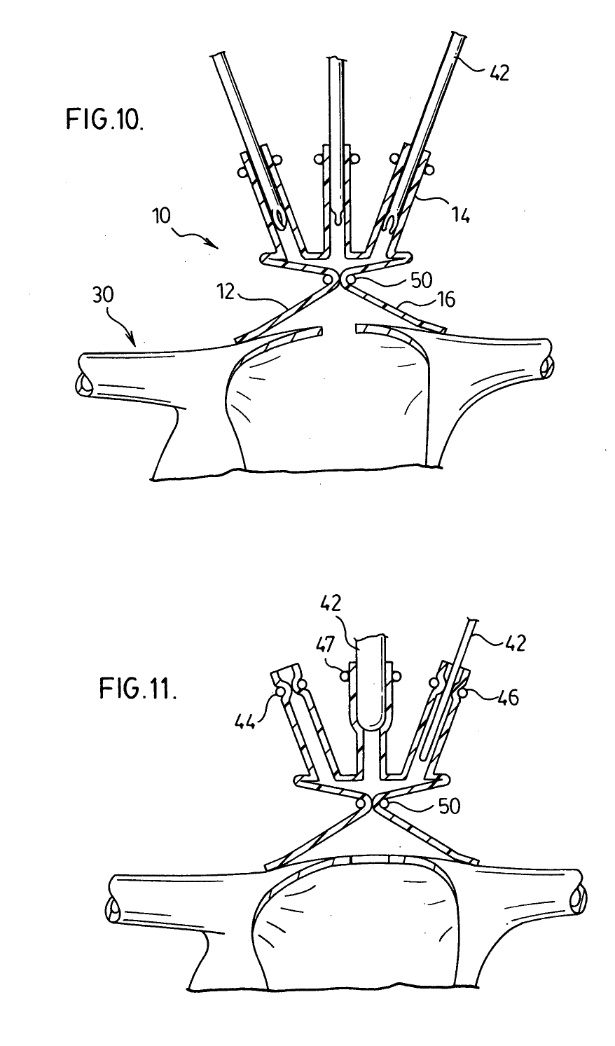

As seen in FIG. 10, the instruments 42 may be withdrawn from the main sleeve and into the branch sleeves 14 and the main sleeve 12 may be closed as by a clamp 50 secured about the exterior wall 16 of the main sleeve 12. As illustrated in FIG. 10, the main conduit through the main sleeve 12 is closed and the branch sleeves 14 are isolated from the heart interior. The various instruments 42 may be removed from the branch sleeves 14 and the branch sleeves exposed to the atmosphere. While in this condition of FIG. 10, additional instruments may be inserted into the branch sleeves. For example, FIG. 11 illustrates a condition in which one of the branch sleeves is closed by a clamp 44 and two different instruments 42 are introduced into two of the branch sleeves to be sealably received therein by clamps 47 and 46. Subsequently, the clamp 50 which is closing the main sleeve 12 may be removed and, once removed, the main conduit of the main sleeve 12 will be open to the branch conduits and the various instruments 42 received within the branch sleeves 14 may then be moved for use within the manifold 10 and within the interior of the heart.

After all of the procedures have been completed, the main conduit of the main sleeve may again be closed with a clamp, the various instruments removed and, in this condition, the side wall of the main sleeve may be collapsed upon each other and secured as by sutures to the outside of the wall of the heart and across the cardiac port 36 so as to close the cardiac port. Thereafter, excess portions of the manifold 10 are cut away.

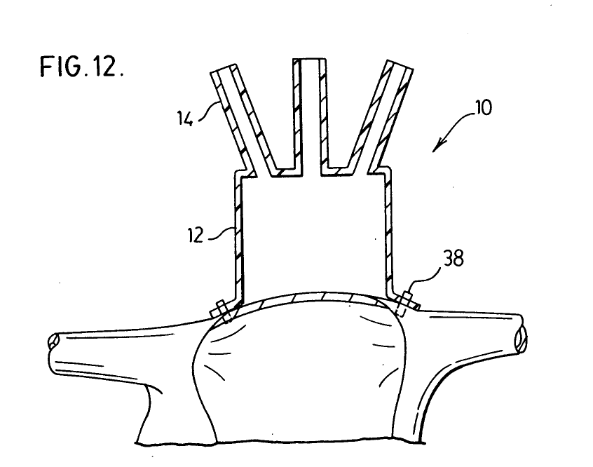

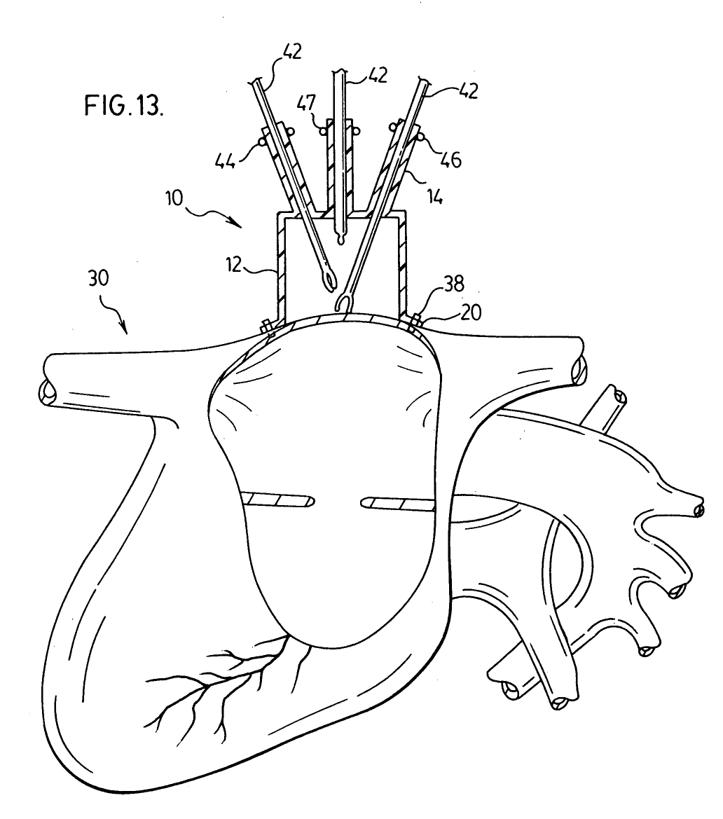

Reference is made to FIGS. 12 and 13 which show an alternate method of use of the manifold in accordance with FIG. 1.

As illustrated in FIG. 12, a heart access manifold 10 as illustrated in FIG. 1 is secured to the exterior surface of the heart by suturing the cuff 20 to the wall of the heart with sutures 38.

Subsequently, as illustrated in FIG. 13, a plurality of instruments 42 are inserted into the branch conduits 14 and sealed therein as by clamps 44, 47 and 46. Subsequently, the instruments 42 are used so as to cut the cardiac port 36 through the wall of the heart. Once a port in the cardiac wall has been opened then, as illustrated in FIG. 8, the instruments 42 can extend into the interior of the heart.

Various arrangements can be provided so as to permit the instruments 42 to be received within the branch sleeves 14, however, in sealed arrangement.

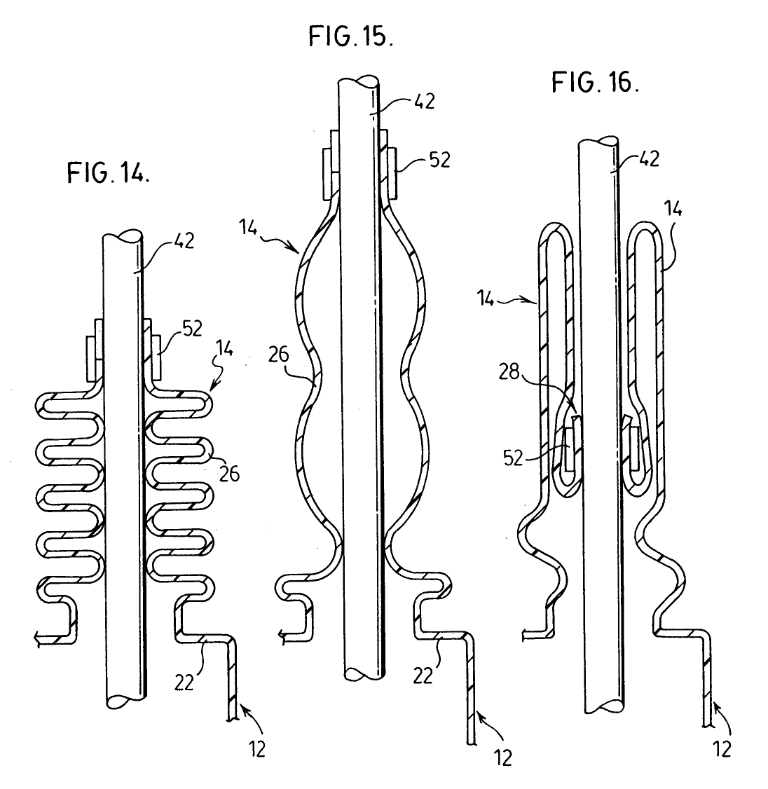

The material which forms the manifold, notably, the side walls of the main sleeve 12 and, particularly the side walls of the branch sleeves 14, preferably is flexible and/or may be provided to have an accordian-like structure which permits the main sleeve 12 or branch sleeve 14 to be contracted or extended as well as to be collapsed and/or to be manipulated to extend in different directions. FIG. 14 illustrates schematically an instrument 42 passing through a branch sleeve 14 with a side wall 26 which is generally biased to assume a compressed or accordian-like configuration. The end of the branch sleeve 14 about the entry port 28 may be secured to the outside surface of the instrument 14 as by a rubber band 52 or other suture or clamping mechanism. The instrument 42 may be moved relative to the main sleeve 12 as by extension or contraction of the branch sleeve 14. FIG. 15 shows the branch sleeve 14 of FIG. 14 in which the instrument 42 has been withdrawn upwardly from the position shown in FIG. 14 by extension of the branch sleeve, however, maintained sealed.

Reference is made to FIG. 16 which illustrates another manner in which a branch sleeve 14 may accommodate a movement of an instrument 42 inwardly and outwardly relative the branch sleeve. In the configuration of FIG. 16, the branch sleeve 14 about the entry port 28 may be secured to the exterior surface of the instrument 42 as by a rubber band or clamp 52. Subsequently, on moving the instrument 42 forward, the entry port 28 may extend downwardly into the branch sleeve 14 with the sleeve doubling back onto itself as illustrated and, thus, permitting the instrument 42 to be maintained in sealed relation yet inserted downwardly into the main sleeve.

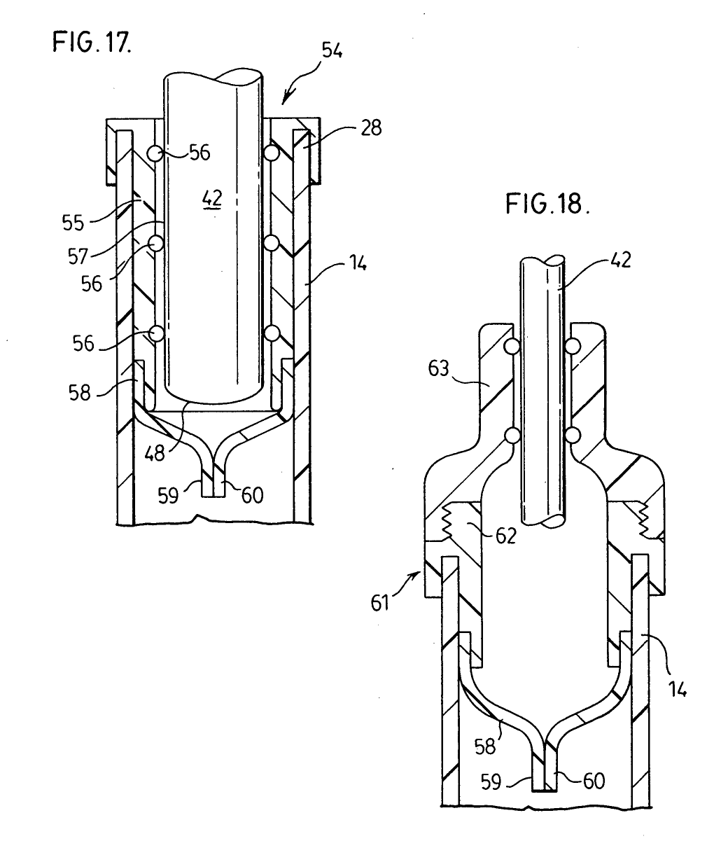

FIG. 17 shows another preferred manner in providing for sliding movement of an instrument 42 within a branch sleeve 14. As seen in FIG. 17, a sealing insert 54 is provided in the entry port 28 to the branch sleeve 14. The sealing insert 54 has a generally cylindrical side wall 55 which carries three elastomeric O-rings 56. The O-rings 56 are sized so as to provide a fluid impermeable seal between the side wall 55 of the insert 54 and an exterior wall 57 of the instrument 42 yet to permit the instrument 42 to slide longitudinally relative to the insert 56 maintaining the sealed relation. The exterior wall 57 of the instrument 42 is shown to be cylindrical and the O-rings 56 are sized so as to provide for sealing engagement between the side wall of the instrument and yet permitting axial sliding of the instrument through the insert.

The insert 54 is also shown as providing at its inner end an elastomeric closure valve 58. The valve is schematically illustrated as comprising an elastomeric bi-valve, that is, an elastomeric member having two flaps 59 and 60 inherently biased into engagement with each other at their interior ends. The valve 58 is shown as being secured at its outer end to an inner end of the insert 54. On an instrument 42 being moved downwardly, a forward end 48 of the instrument 42 will engage the elastomeric valve 58 and urge the flaps outwardly. Thus, the instrument 42 may pass downwardly through the valve 58. The valve 58 also permits the instrument 42 to be slid axially therethrough. On the instrument 42 being withdrawn upwardly pass the valve 58, the inherent resiliency of the valve flaps effectively closes the branch sleeve 14 against blood flow therepast. The valve 58 is preferably selected such that it will effectively seal a branch sleeve 14 under the pressures experienced in the heart.

Reference is made to FIG. 18 which shows a modified form of the sealing insert shown in FIG. 17 in which the branch sleeve 14 carries at its end a rigid insert 61 which carries a closure valve 58 as in FIG. 17. The rigid insert 61 is provided with male threaded end portion 62 adapted to receive a female threaded end cap 63 which carries O-rings 56 sized to receive an instrument 42 therein. It is to be appreciated that by removal of the end cap 63, different other caps may easily and readily be applied as to carry different sized instruments and with the instruments capable of being removed and inserted while the forward end of the branch conduit 14 remains under heart pressure.

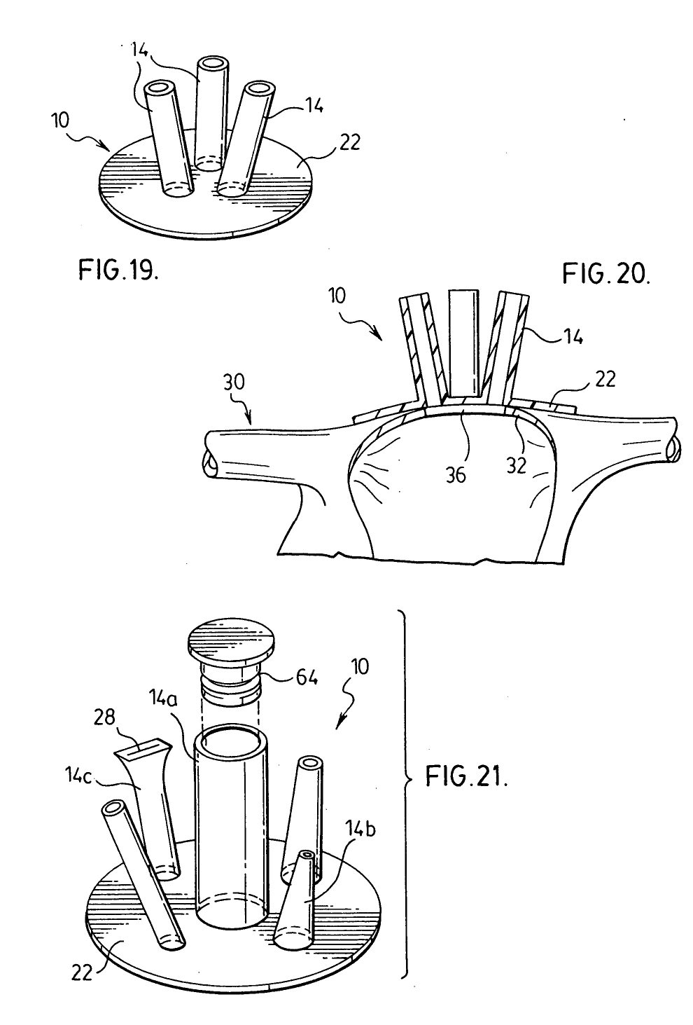

Reference is made to FIGS. 19 and 20 which show a second embodiment of a heart access manifold 10 in accordance with the present invention. The embodiment of FIG. 19 is similar to that in the embodiment of FIG. 1 in having a distribution disc 22 which has a number of openings therethrough with each opening having a branch sleeve 14 sealably secured thereto. The distribution disc 22 is secured directly to the wall 32 of the heart 30 as illustrated in FIG. 20. If necessary, access can be gained to the outside surface of the wall 32 of the heart by deflecting the distribution disc 22 upwardly.

Reference is made to FIG. 21 which shows a third embodiment of a heart access manifold 10 not dissimilar to that shown in FIGS. 19 and 20, however, illustrating a number of different configurations for branch sleeves 14. In FIG. 21, a main centered branch sleeve 14a is illustrated as being provided with a simple closure plug 64 which may preferably be rigid and may have protrusions on its outside surface to assist in securing the same within the branch sleeve 14a as by a rubberized band or clamp.

Another of the branch sleeve 14b is illustrated as tapering upwardly as a coil as it extends from the distribution disc 22. By tapering upwardly, increased movement may be provided and, as well, this branch sleeve 14b may be adapted to secure about relatively small sized instruments.

A branch sleeve 14c is illustrated as being provided with its access port 28 closed by the end of the branch sleeve 14c being sealed closed upon itself as by adhesives. For use, the branch sleeve 14c may merely be cut below the place where its entry port 28 is sealed.

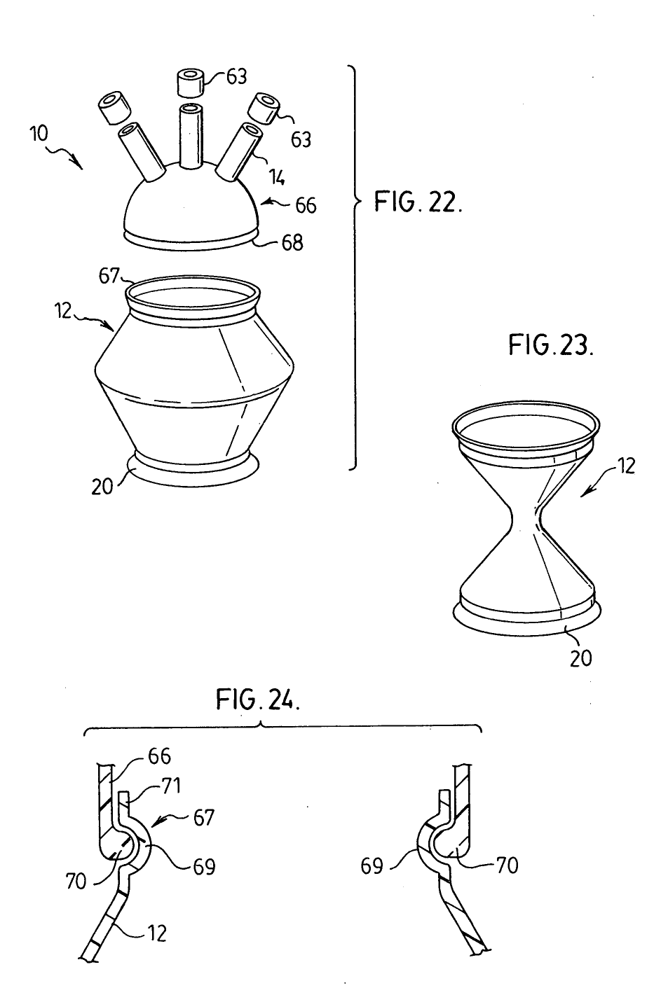

Reference is made to FIGS. 22 to 24 which illustrate a fourth embodiment of a heart access manifold 10 in accordance with the present invention. The manifold is illustrated as comprising two main parts, namely a main sleeve portion 12 and a distribution cap 66. The main sleeve portion 12 has an entry end opening 67 which is adapted to removably sealably engage with an exit end opening 68 of the distribution cap 66. As shown, the distribution cap 66 is a substantially domed member which is closed at an upper end by distribution wall 22 but for branch exit ports with each branch exit port open to a branch sleeve 14. The distribution cap 66 is adapted to be applied to and to be removed from the main sleeve portion 12. FIG. 24 schematically illustrates one mechanism for coupling the main sleeve portion 12 to the distribution cap 66 in which the main sleeve portion 12 carries about its entry end opening 67, an outwardly directed annular U-shaped channel 69 which is adapted to receive therein an inwardly extending annular rib 70 about the exit end opening 68 of the distribution cap 66. An upwardly extending annular vane 71 is provided at the upper end of the main sleeve portion 12 adapted to be urged by pressure inside the manifold 10 into sealing engagement with the inside surfaces of the distribution cap.

The preferred main sleeve portion illustrated in FIG. 22 is adapted to assume either an open, expanded configuration as illustrated in FIG. 22 or a closed, collapsed configuration as illustrated in FIG. 23. When the side wall 16 of the main sleeve portion 12 may comprise a simple fabric, then the main sleeve portion 12 may be caused to assume and maintain the closed configuration as, for example, by a simple purse-string suture or other clamping device. The main sleeve portion 12 may carry as an element thereof, a clamping device such as a flexible string or belt carried in loops which can be pulled to facilitate fast and easy closing of the main sleeve portion. The side wall 16 of the main sleeve portion 12 could be resilient and inherently biased such that they either assume an open condition as illustrated in FIG. 22 or a closed condition as illustrated in FIG. 23.

In the main sleeve portion 12 of FIG. 22 about the entry end opening 67, an annular portion there may be a substantially resilient elastomeric ring which will maintain its circular shape and thus assist in, for example, coupling of the entry end opening 67 of the main sleeve portion 12 to the exit opening 67 end of the distribution cap 66. Similarly, an annular portion of the main sleeve portion 12 about or near the cuff 20 may be a similar resilient ring with the cuff 20 to extend downwardly from such a substantially rigid or elastomeric annular end portion.

FIG. 22 illustrates the distribution cap 66 as including branch sleeves 14 which extend upwardly and carry at their end, a removable end cap 63 as, for example, illustrated in FIG. 18.

For surgery on a beating heart, in certain circumstances, be advantageous to position the manifold 10 and/or heart port 36 and/or to assist in maintaining the wall of the heart above the cardiac port 36 from undue movement.

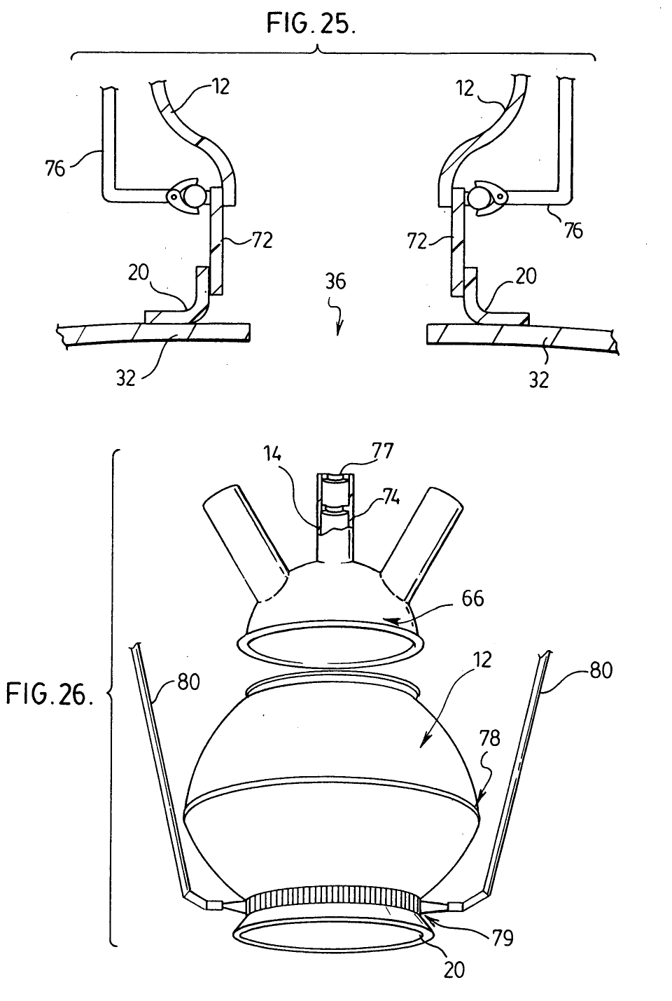

FIG. 25 illustrates a lowermost portion of a heart access manifold 10 in accordance with the present invention and showing, as is the case with the other embodiments, a cuff 20 as secured to the wall 32 of the heart. The cuff 20 is connected to a lower end of an elastomeric annular ring 72 having its upper end coupled to the main sleeve portion 12 and also secured to an upper end of the ring 72. The annular ring is a rigid annular loop 75 of torroidal shape. Two securing arms 76 hold the loop 75 at diametrically opposed locations. The securing arms extend upwardly and are adapted to be secured relatively fixedly by their upper end so that the loop 75 is relatively rigidly held at two diametrically opposed positions by the securing arms 76. The annular loop 75 and, thus, the upper end of the elastomeric ring 72 as well as the lower end of the main sleeve 12 are held constrained against movement.

The elastomeric ring 72 is resilient and can stretch and contract to assist in accommodating relative movement of the wall of the heart relative to the rigid ring and the main sleeve.

Reference is made to FIG. 26 which shows a further embodiment of heart access manifold 10 in accordance with the present invention. The embodiment illustrated has similarities to the embodiment illustrated in FIG. 22 insofar as it comprises a separate main sleeve portion 12 and a separate distribution cap 66 adapted to be removably secured together. Each branch sleeve 14 of the distribution cap 66 has a pair of resilient rubber washers 77 therein to frictionally engage and form a seal with instruments to be inserted.

The main sleeve portion 12 is shown to have an equatorial band 78 extending circumferentially about its center and is adapted to carry a clamping device or other closure device to close the main sleeve portion 12.

At the exit end of the main sleeve portion 12, there is provided a relatively rigid annular band 79 secured about the cuff 20 and adapted to be held at diametric locations by elongate holder arms 80 to constrain the main sleeve portion 12 against movement. The cuff 20 is shown as extending downwardly from the rigid band 79 and adapted to be coupled to the wall of the heart. The rigid band is to be used to anchor a robotic device.

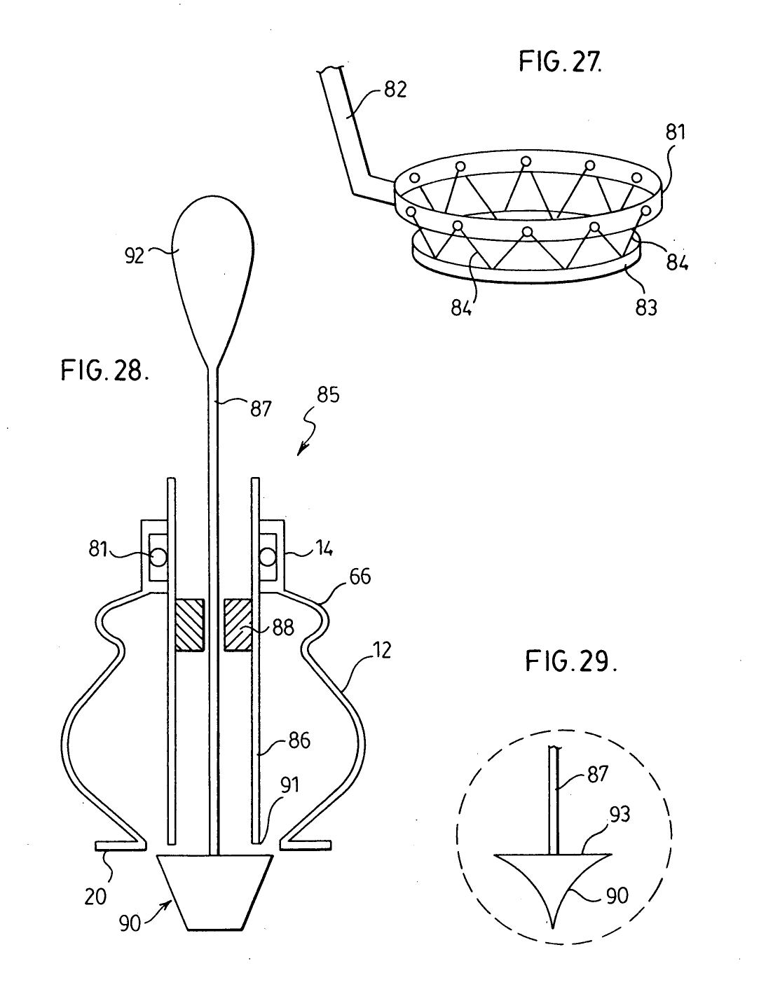

FIG. 27 illustrates another arrangement of the rigid annular band 79 of FIG. 26. FIG. 27 shows a rigid annular ring 81 secured at one side to a rigid elongate holder rod 82 which extends upwardly away from the ring 81. A cuff engagement flange 83 is adapted to either form the cuff of a main sleeve portion or to be secured to a cuff of the main sleeve portion. The rigid ring 81 and the cuff engagement flange 83 are coupled together by a set of V-shaped strings 84 to provide support but some flexibility.

Reference is made to FIGS. 28 to 34 which illustrate one specialized surgical instrument comprising a punch tool 85 adapted to be used in conjunction with a heart access manifold 10 in accordance with the present invention. FIG. 28 schematically shows a heart access manifold 10 in cross-section having a single branch sleeve 14 extending upwardly from a distribution cap 66 secured to a main sleeve portion 12. The punch tool 85 has a hollow cylindrical tube 86 which is received in sealed engagement with an O-ring 81 inside of the branch sleeve 14. Inside of the hollow tube 86, there is provided an elongate shaft 87 slidable in the tube 86 by being received within locating plug 88 which coaxially locates the shaft 87 within the tube 86 and, as well, provides a seal. At the upper end of the shaft 87, there is provided a handle 92 permitting manipulation by a surgeon. At the lower end of the shaft 87, there is provided a cutting blade 90 which is seen in one side view in FIG. 28 and in cross-section normal thereto in FIG. 29. The forward end of the tube 86 is sharpened and forms a circular knife 91.

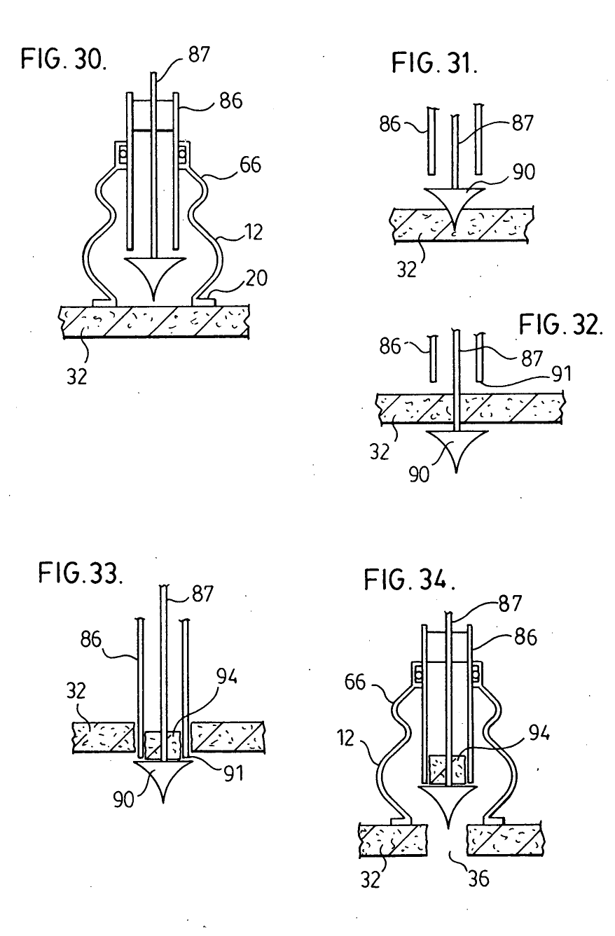

Use of the punch tool is schematically illustrated in sequence in FIGS. 30 to 34.

As seen in FIG. 30, the cutting blade is above the wall 32 of a heart and is urged downwardly through the wall as depicted in FIG. 31 and FIG. 32. In FIG. 32, the blade 90 has passed through the wall of the heart. A rearwardly directed surface 93 of the blade 90 provides a cylindrical platen upon which the knife 91 of the tube 86 may then be forced downwardly as illustrated in FIG. 33 so as to cut a circular plug 94 from the side wall of the heart forming the heart part 36. Subsequently, the circular plug 94 may be moved upwardly by movement of the entirety of the blade 90 and the tube 86.

Subsequently, the punch tool 85 may be moved rearward of the main sleeve portion 12. The main sleeve portion 12 may be closed and the distribution cap 66 may be removed and replaced by another distribution cap 66 carrying instruments suitable for carrying out surgery within the interior of the heart.

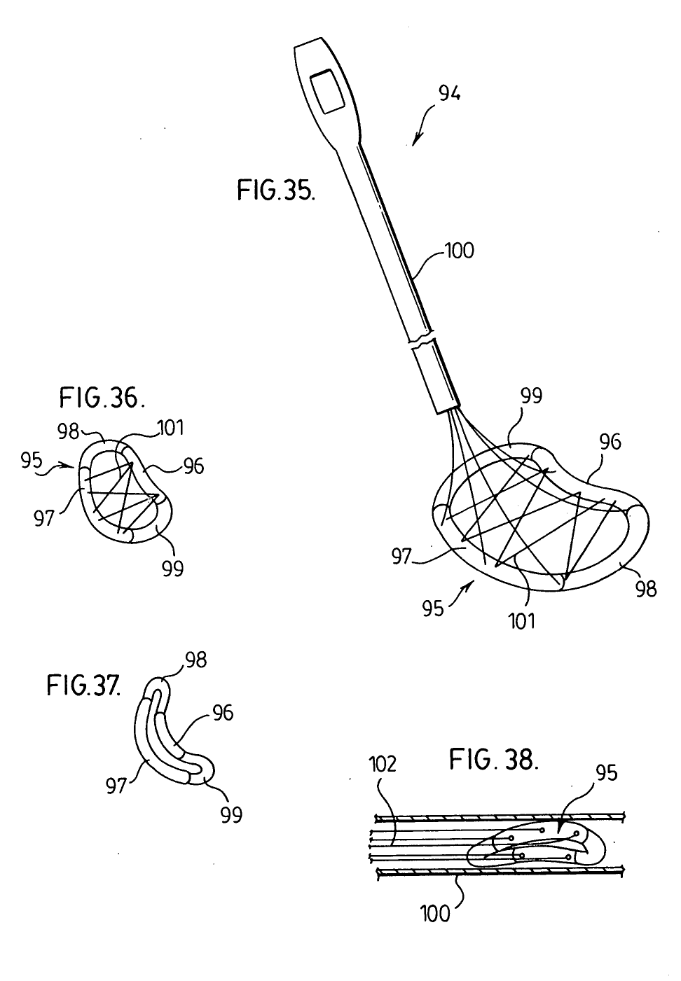

Reference is made to FIGS. 35 to 40 illustrating another specialized tool 94 and its use. The tool 94 comprising a Mitral valve repair instrument and it is adapted to apply a Mitral valve repair device 95.

FIGS. 36 and 37 best illustrate the Mitral valve repair device 95 comprising a ring formed of two semi-rigid segments 96 and 97 alternating with two flexible segments 98 and 99. The ends of the four segments are connected together. By reason of the flexible segments 98 and 99, the ring can be bent and deformed so as to fit within an introducer tube 100 forming part of the tool 94. The repair device 95 includes a number of cross strings 101 which form a support net for the Mitral valve leaflets, thus preventing the leaflets from pro-lapsing beyond the plane at the Mitral valve annulus and thereby preventing Mitral valve regurgitation. For ease of illustration, in FIGS. 37, 38 and 39, the strings 101 of the support net for the Mitral valve repair device are not shown. As shown in FIG. 38, the ring of the device 95 is collapsed and inserted inside the cylindrical introducer tube 100 of the tool 94 with a plurality of positioning tubes 102 attached to the different segments of the ring of the device 95.

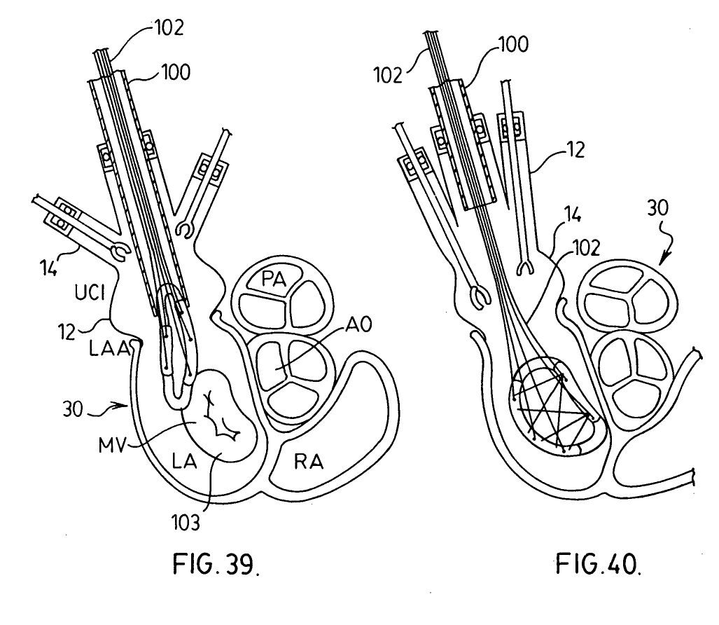

As FIG. 39 illustrates, the tube of tool 94 introduced into the interior of a heart via the left atrium via an orifice cut into the left atrial appendix and the Mitral valve repair device 95 being pushed out of the introducer tube 100 by the tubes 102. The device 95 is positioned over the Mitral valve 103 with the ring about the valve 103. Subsequently, two other instruments 42 are introduced into the left atrium to secure the ring of the device 95 about the valve 103. Manoeuvring of the ring and the various instruments may be carried out under image guidance and with possible robotic assistance. Once the ring of the device 95 is suitably attached, then the tubes 102 that help to position and to hold the ring will be severed proximate the ring.

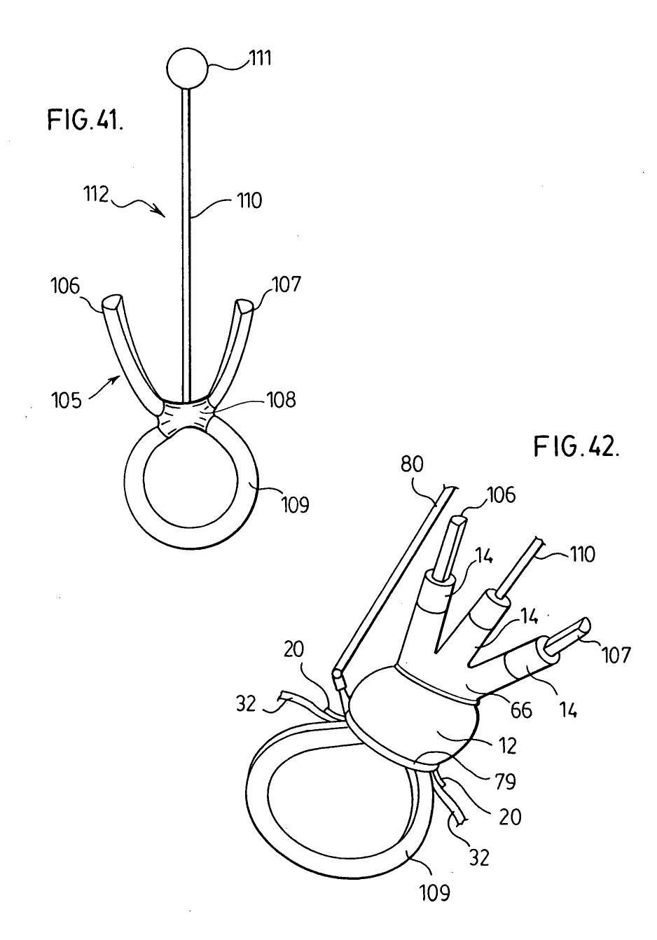

FIG. 41 shows an Intracardiac loop instrument 112 comprising an elongate member 105 having two discrete ends 106 and 107. Each end is passed through a central harness 108 such that a loop 109 is formed on one side of the harness 108 and the two distal ends 106 and 107 of the loop 109 extend from the other side of the harness 108. A loop holder shaft 110 extends from the harness 108 to a handle 111.

FIG. 42 illustrates the loop instrument 112 as used in association with a heart access manifold 10 in accordance with the present invention. As illustrated, the loop 109 extends into the interior of the heart through the cardiac port with the harness (not shown) substantially within the main sleeve portion 12 and with each distal end 106 and 107 of the loop extending out of separate spaced branch sleeves 14 and with the handle 110 extending out of a central of the branch sleeve 14. The handle 110 may preferably be connected to a robotic arm for manipulation. FIG. 42 also illustrates a holder rod 80 adapted to be connected outside the heart to a robotic arm. The rod 80 is coupled at its lower end to be coupled to a rigid loop 79 secured about the cuff 20 of the heart access manifold 10.

Referring to FIGS. 41 and 42, the loop 109 preferably comprises a microwave Ablation device manufactured by AFx Inc. In use, the loop 109 is first made as small as possible such that it can be inserted into the distribution cap 66 and the distribution cap 66 secured to a closed main sleeve portion 12. Subsequently, the main sleeve portion 12 is opened and the loop 109 is then extended down through the main conduit and into the interior of the heart. Subsequently, the size of the loop 109 is enlarged as by urging each distal end 106 and 107 of the loop 109 to slide downwardly into the interior of the heart. Ablation energy may be applied as required.

Other useful surgical instruments would include suturing devices.

The preferred embodiment illustrated in FIGS. 1 to 3 shows branch sleeves 14 as extending from the distribution plate 22. FIG. 1 shows in dotted lines an additional branch sleeve 14 illustrating the branch sleeves may also, for example, emanate from the cylindrical side wall 16.

While the main sleeve portion 12 has been shown in many embodiments as having a cylindrical side wall, it is to be appreciated that this is not limiting. Not only is there no need for the main sleeve portion as, for example, in some of the embodiments, it is appreciated that the relative shape and configuration of the main sleeve portion in each of the branch sleeve portions may vary widely without departing from the scope of the invention.

While the invention has been described with reference to preferred embodiments, many modifications and variations will now occur to persons skilled in the art. For a definition of the invention, reference is made to the following claims.

1. A heart access manifold having an interior within a manifold wall, an exit port through the manifold wall and at least one entry port through the manifold wall,
the exit port being adapted to sealably engage about an opening in a wall of the heart for communication with the interior of the heart to place the interior of the manifold to be under the same pressure as the interior of the heart,
the at least one entry port providing access through the manifold wall into communication with the interior of the heart via the exit port, each entry port sealable to maintain pressure on the interior side of the manifold,
each entry port adapted for passage therethrough to the interior of the manifold of at least one implement while maintaining pressure in the interior of the manifold.

2. A manifold as claimed in claim 1 wherein the manifold wall has a main sleeve portion defining a main conduit providing communication from the exit port to each entry port.

3. A manifold as claimed in claim 2 including a closure mechanism to sealably close the main conduit against communication therethrough from the exit port to the entry ports.

4. A manifold as claimed in claim 1 having, for each entry port, a branch sleeve portion defining a branch conduit for communication from the entry port into the interior of the heart.

5. A manifold as claimed in claim 4 including a closure mechanism to sealably close each branch sleeve portion against communication therethrough from the entry port to the interior of the manifold.

6. A manifold as claimed in claim 2 wherein the manifold wall having, for each entry port, a branch sleeve portion defining a branch conduit providing communication from the entry port into the main conduit.

7. A manifold as claimed in claim 6 including a closure mechanism to sealably close the main conduit against communication from the exit port to each branch sleeve portion.

8. A manifold as claimed in claim 7 including a closure mechanism to sealably close each mouth sleeve portion against communication therethrough.

9. A manifold as claimed in claim 3 wherein the main sleeve portion having an exit end disposed about the exit port and an entry end opening to the branch sleeve portion, the entry end closed by a distribution wall having one branch port therethrough for each branch sleeve portion,
each branch sleeve portion having an exit end disposed about a respective branch port and an entry end disposed about its respective entry port.

10. A manifold as claimed in claim 1 wherein the main sleeve portion is collapsible to sealably close communication through the main conduit from the exit port to the entry ports.

11. A manifold as claimed in claim 7 wherein each branch sleeve portion is collapsible for sealably closing the branch sleeve portion to prevent communication therethrough.

12. A manifold as claimed in claim 1 including a cuff about the exit port for sealable engagement with the wall of the heart about the opening.

13. A manifold as claimed in claim 12 wherein said cuff is adapted to be secured by situres about the opening in the wall of the heart.

14. A manifold as claimed in claim 1 wherein the implement is slidable received in one relative to the entry port maintaining a sealed relation therein to permit insertion and withdrawal of interior portions of an implement into and out of the interior of the manifold and the interior of the heart.

15. A manifold as claimed in claim 9 wherein the distribution wall is removably sealably coupled to the entry end of the main sleeve portion for replacement by the same or a similar distribution wall.

16. A manifold as claimed in claim 15 when the distribution wall portion can be removed from and coupled to the entry end of the main sleeve portion while the main conduit is maintained closed by the main conduit closure mechanism.

17. An access manifold as claimed in claim 1 wherein the main sleeve portion includes a flexible portion extending fully circumferentially about the main conduit and, to at least some extent, longitudinally of the main conduit to provide for relative movement of segments of the main sleeve portion on either side of the flexible portion.

18. An access manifold as claimed in claim 17 wherein each branch sleeve portion includes a flexible portion extending fully circumferentially about the branch conduit and to, at least some extent, longitudinally of the branch conduit to provide for relative movement of segments of each branch sleeve portion on either side of the flexible portion.

19. A manifold as claimed in claim 1 wherein the manifold wall comprises a flexible material with the manifold wall being collapsible as to close communication through the manifold and with the manifold being inflatable under pressure from the interior of the heart through the heart opening and the exit port is sealed and engaged about the opening in the wall of the heart when the entry ports are closed.

20. A manifold as claimed in claim 19 wherein the manifold wall comprises a flexible fabric impervious to blood.

21. A manifold as claimed in claim 1 wherein each implement includes a head at an inner end thereof and an elongate stem extending from the head through the entry port for coupling to a control mechanism exterior of the manifold, the implement being movable within the interior of the manifold and/or within the interior of the heart while maintaining a sealed engagement between the entry port and the stem of the implement.

22. A manifold as claimed in claim 21 wherein the stem of the implement is slidably received within the entry port in sealed relation therewith.

23. A manifold as claimed in claim 22 wherein the entry port is fixedly secured in sealed relation to an exterior surface of the stem and the flexibility of the manifold wall accommodates relative movement of the implement head within the manifold interior and the heart interior.

24. A manifold as claimed in claim 1 including a support ring secured to the manifold disposed about the exit port proximate to the exit port and adapted to be held in a relatively fixed relation.
